(19) United States
(12) Patent Application Publication
(10) Pub. No.: US 2022/0142802 A1
(43) Pub. Date: May 12, 2022

(54) PROTECTIVE BOOT

(71) Applicant: Walgreen Health Solutions, Lake Forest, IL (US)

(72) Inventors: Charles R. Walgreen, Lake Forest, IL (US); Sakeena I. Haq, Lake Forest, IL (US)

(21) Appl. No.: 17/096,345

(22) Filed: Nov. 12, 2020

(51) Int. Cl. A61F 5/01

(52) U.S. Cl. CPC A61F 5/0113

(57) ABSTRACT

A protective boot for supporting a leg of an individual and providing leg and foot support in a manner capable of reducing the risk of foot drop, heel pressure ulcers, and other foot conditions. The boot has a continuous cavity within leg and forefoot portions of the boot and complementary in size and shape to receive and support the lower leg and foot of the individual while supporting the foot in an upright position. A forefoot flap spans a portion of an anterior opening in the forefoot portion, and a strap traverses an exterior surface of the forefoot flap so that the forefoot flap contacts the dorsum of the foot, separates and protects the dorsum from the strap, distributes pressure applied by the strap to the dorsum and, in combination with a distal region of the forefoot portion, resists movement of the foot in the dorsiflexion and plantarflexion directions.

### BACKGROUND OF THE INVENTION

The present invention generally relates to devices adapted to support a leg of an individual while reclining, especially patients that are bedridden and as a result are prone to bed sores, mild to moderate foot drop (equinus deformity foot-ankle), and other conditions that can affect the foot.

Bedridden individuals can suffer from a variety of conditions brought on by being confined in bed, especially if limited to the supine position. For example, bedridden individuals are susceptible to heel pressure ulcers, foot drop caused by pressure over the peroneal nerve, relaxation and weakening of the muscles controlling the foot, and heel cord contracture. As a preventive measure, various foot support devices have been proposed, notable examples of which are disclosed in U.S. Pat. No. 4,186,738 to Schleicher et al. and U.S. Pat. Nos. 5,449,339, 7,458,948, and 10,334,905 to Drennan.

The Drennan patents disclose heel-supporting boots commercially available from Walgreen Health Solutions, LLC, under the name HEELIFT® Suspension Boot. The boots include a unitary body formed of a flexible and compressible material that defines a foot supporting portion and a leg supporting portion. The leg supporting portion is configured to wrap around the leg of an individual away from the individual's foot, and is equipped with adjustable straps that secure the boot in place on the individual's leg to inhibit movement of the boot out of a proper supportive position in the event the individual moves. The leg supporting portion supports the individual's leg from beneath to suspend the individual's foot above the surface on which the individual is reclined, offloading such areas as the back of the heel, the Achilles tendon, and ankle bones with the result that heel ulcers can be prevented and pre-existing wounds can heal faster.

In addition, the leg supporting portion elevates the calf relative to the bed to remove pressure from the peroneal nerve at the upper end of the leg. The foot supporting portion supports the foot by applying pressure to the sole, thereby preventing foot drop, heel cord contracture, etc. The posterior surface of the boot may be provided with a friction-reducing element that promotes free sliding movement of the boot over the bed surface, and a stiffener may be provided within the boot body to inhibit buckling and folding of the boot due to friction with the bed surface.

While having a number of beneficial elements, foot support devices in the prior art may allow for excess room for the foot within the device, allowing the foot to shift and rotate within the device.

### BRIEF DESCRIPTION OF THE INVENTION

The present invention provides a protective boot adapted to support a leg of a human while reclining and further adapted to provide leg and foot support in a manner capable of reducing the risk of foot drop, heel pressure ulcers, and other foot conditions.

According to one nonlimiting aspect of the invention, a protective boot includes a shell formed of a flexible and compressible material. The shell has a proximal leg portion, a distal forefoot portion contiguous with and projecting from the leg portion in a transverse direction thereto, a continuous cavity defined by and within the leg and forefoot portions and complementary in size and shape to receive and support the lower leg and the foot of the individual wearing the boot while supporting the foot of the individual in an upright position, an anterior opening defined in the leg and forefoot portions to permit the foot and lower leg of the individual to pass therethrough into the cavity within the leg and forefoot portions, left and right leg lateral regions defined by the leg portion and separated by a first portion of the anterior opening, and left and right forefoot lateral regions defined by the forefoot portion and separated by a second portion the anterior opening. The boot further includes a closure system comprising at least a first strap that adjustably closes the first portion of the anterior opening with the left and right leg lateral regions of the leg portion and at least a second strap that adjustably draws the forefoot lateral regions of the forefoot portion toward each other across the second portion of the anterior opening so as to enable the forefoot lateral regions of the forefoot portion to contact and support lateral sides of the foot of the individual when present within the cavity and resist movement of the foot in the pronation and supination directions thereof but provide only minimal if any support to the dorsum of the foot. A forefoot flap spans the second portion of the anterior opening, and the second strap of the closure system traverses an exterior surface of the forefoot flap so that the forefoot flap is enabled to contact the dorsum of the foot when present within the cavity, separate and protect the dorsum of the foot from the second strap, distribute pressure applied by the second strap to the dorsum and, in combination with a distal region of the forefoot portion, resist movement of the foot in the dorsiflexion and plantarflexion directions.

According to another nonlimiting aspect of the invention, a method of using the protective boot described above includes receiving and supporting the lower leg and the foot of the individual within the continuous cavity within the leg and forefoot portions so as to support the foot of the individual in an upright position within the forefoot portion, spanning the second portion of the anterior opening and the dorsum of the foot with the forefoot flap, and traversing the exterior surface of the forefoot flap with the second strap of the closure system and drawing the left and right forefoot lateral regions toward each other with the second strap so that the second strap applies pressure to the forefoot flap and the forefoot flap contacts the dorsum of the foot, separates and protects the dorsum of the foot from the second strap, distributes the pressure applied by the second strap to the dorsum and, in combination with the pressure applied to the sole of the foot by the distal region of the forefoot portion, resists movement of the foot in the dorsiflexion and plantarflexion directions.

A technical effect of the invention is the ability of the boot to provide support to the foot within the device, while limiting the ability of the foot to shift and rotate within the device by providing resistance to movement of the foot in the dorsiflexion and plantarflexion directions. The addition of the forefoot flap also protects the foot and toes from injury and from foreign materials lodging between the toes when the individual is in a prone or supine position. Furthermore, the addition of the forefoot strap helps to secure the boot so that the foot/ankle do not move within the boot, thereby reducing the risk of injury to the skin from shear and friction forces.

Other aspects and advantages of this invention will be appreciated from the following detailed description.

### BRIEF DESCRIPTION OF THE DRAWINGS

FIG. 1 is a perspective view of a protective boot adapted to support a human leg in accordance with a nonlimiting embodiment of the invention.

FIGS. 2 and 3 are, respectively, right and left side views of the protective boot shown in FIG. 1, and FIG. 4 is a top view of the protective boot shown in FIG. 1.

FIG. 5 is a cross-sectional view of the protective boot shown in FIG. 1.

FIG. 6 schematically represents a cross-sectional view of the protective boot shown in FIG. 1, with a human foot located and supported within the boot.

### DETAILED DESCRIPTION OF THE INVENTION

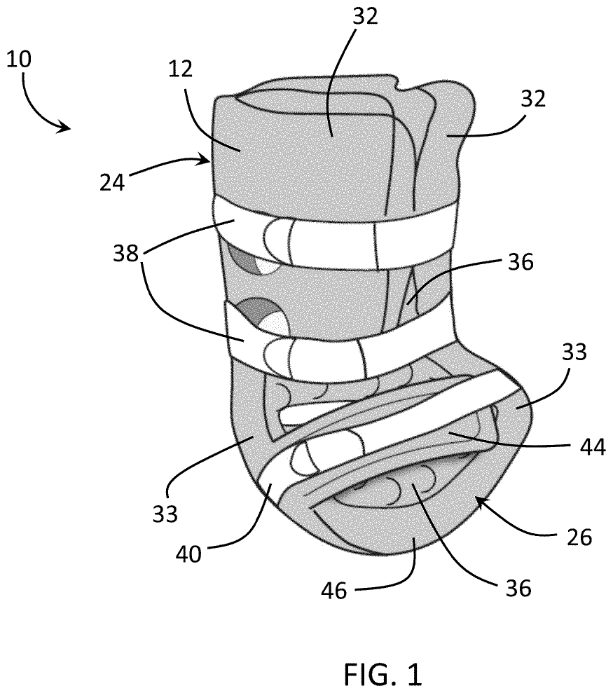

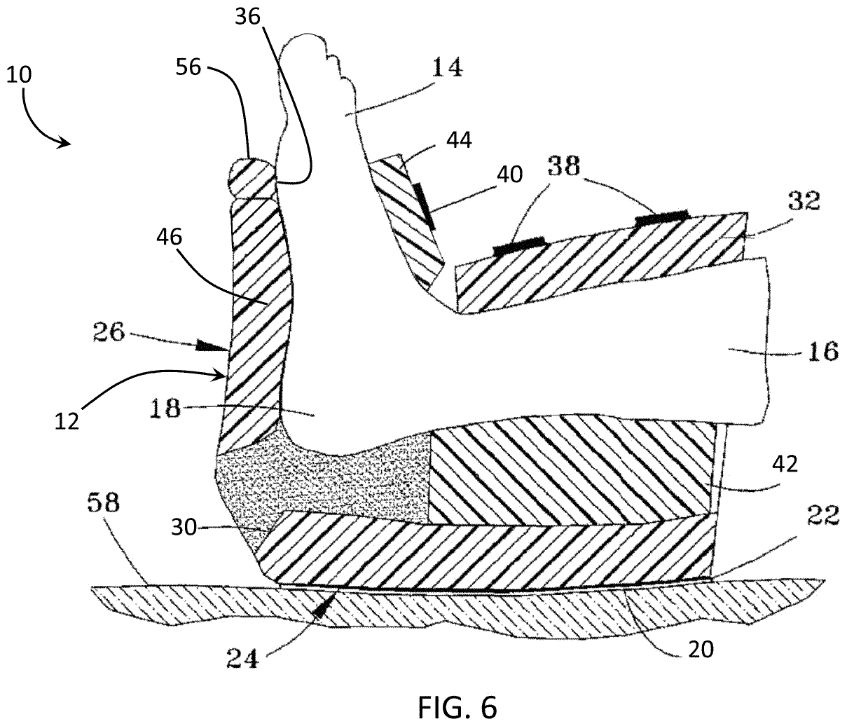

FIGS. 1 through 6 represent various views of a protective boot 10 configured to be worn on a human foot 14 and lower leg 16 (FIG. 6) while the individual is in a generally supine position (reclining) or prone position (face down) on a bed or any other surface on which an individual might recline while wearing the boot 10. The boot 10 generally has a proximal leg portion 24 and a distal foot portion 26 contiguous with and projecting from the leg portion 24 in a transverse direction thereto. The leg and foot portions 24 and 26 are adapted to receive an individual's leg 16 and foot 14, respectively, when the individual is wearing the boot 10. The boot 10 is configured to support the individual's lower leg 16 and heel 18 in a manner that suspends the heel 18 to avoid foot drop and relieves heel pressure to avoid heel pressure ulcers, while also firmly securing the foot 14 in a manner that limits the ability of the foot 14 to shift and rotate within the boot 10, including the ability to resist movement of the foot 14 in the dorsiflexion and plantarflexion directions, as well as the pronation and supination directions.

To facilitate the description provided below of the embodiment represented in the drawings, relative terms, including but not limited to, “proximal,” “distal,” “anterior,” “posterior,” “vertical,” “horizontal,” “lateral,” “front,” “rear,” “side,” “forward,” “rearward,” “upper,” “lower,” “above,” “below,” “right,” “left,” etc., may be used in reference to the perspective of an individual while wearing the boot 10. All such relative terms are intended to indicate the construction and use of the boot 10, and therefore help to define the scope of the invention.

The nonlimiting embodiment of the boot 10 shown in the drawings comprises a soft foam shell 12 and a closure system that includes straps to secure the shell 12 to the lower leg 16 and foot 14. The shell 12 is preferably a unitary, one-piece body formed of a flexible, compressible foam material, more preferably a material that is capable of being heated and sterilized in an autoclave or oven with limited shrinkage. A particularly preferred shell 12 is formed from a slab of open-cell non-allergenic resilient foam material such as polyurethane foam, with sufficient thickness to elevate an individual's foot 14 and lower leg 16 above a bed and provide sufficient structural support to inhibit movement of the foot 14 and lower leg 16 within the boot 10. If formed to have a foam construction, the shell 12 has a soft high-friction foam surface that may be smooth or convoluted, the latter comprising a pattern of peaks and valleys as represented in FIGS. 1, 3, and 5. Commercially available foam materials having this type of surface are known as convoluted foam or egg crate foam.

The shell 12 defines a continuous cavity 34 within the boot 10 that is complementary in size and shape to receive the foot 14 and lower leg 16 of an individual. A low-friction and nonabsorbent inner lining material may overlie the interior surface of the shell 12 within the cavity 34. As used herein, “low friction” refers to a material that provides a surface that has a lower friction than would be the case in the absence of the material, and in particular lower than the foam surface of the shell 12. Such an inner lining material may also be nonabsorbent, and in particular less absorbent than the foam material of the shell 12, to provide a nonabsorbent and sanitary interface between the boot 10 and the skin of an individual's foot 14. Such an inner lining material may also be water repellant, which in the case of an individual with an exudatous wound inhibits exudate from the wound from being absorbed into the shell 12, thus providing a level of infection control. The inner lining material may cover the interior surface of the shell 12 in its entirety and be permanently bonded (e.g., laminated) to the foam surface of the shell 12, washable to remove exudate and other residues from its surface, and sufficiently soft to maintain a comfortable interface between the shell 12 and the individual's skin over long periods of time, while also providing a level of toughness to prevent tears that would expose the underlying absorbent foam surface of the shell 12. Various low friction and nonabsorbent materials are foreseeable for use as an inner lining material.

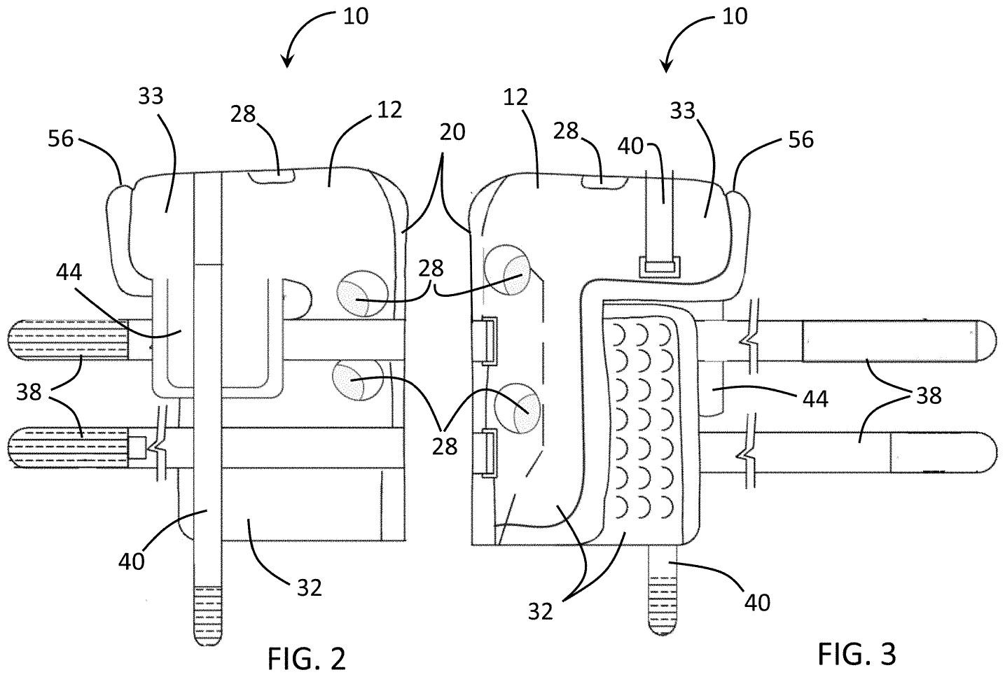

Similar to an inner lining material on the interior surface of the shell 12, the exterior surface of the shell 12 may be partially or entirely covered with one or more low friction materials. As a nonlimiting example, a low-friction exterior surface 20 (FIGS. 2, 3, 5, and 6) located at the posterior external surface of the leg portion 24 of the boot 10 facilitates motion of the boot 10 relative to its surrounding environment, for example, bed coverings beneath while the wearer is in bed, while simultaneously inhibiting motion of the individual's foot 14 and ankle within the boot 10, thereby reducing the likelihood of unintended displacement of the foot 14 within the boot 10. The exterior surface 20 may be defined by a fabric material or a backplate applied to the exterior surface of the shell 12.

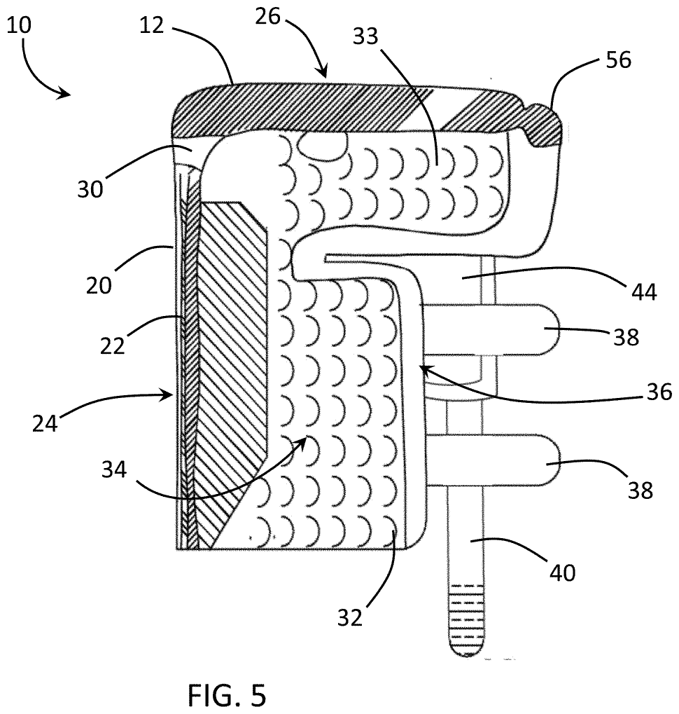

The boot 10 can optionally incorporate a stiffener 22. As shown in FIGS. 5 and 6, the stiffener 22 can be located within at least the posterior region of the leg portion 24, and may extend to include a distal region 46 of the forefoot portion 26 to minimize buckling of the boot 10 within the leg and forefoot portions 24 and 26. Particularly suitable materials for the stiffener 22 are capable of being heated and sterilized in an autoclave or oven with limited shrinkage.

The interior and exterior surfaces of the boot 10 are separated from each other by a continuous rim 56 that completely borders an anterior opening 36 (FIGS. 5 and 6) to the cavity 34, including oppositely-disposed (left and right from the perspective of an individual while wearing the boot 10) lateral regions 32 of the leg portion 24 that extend from a posterior region of the leg portion and oppositely-disposed (left and right from the perspective of an individual while wearing the boot 10) lateral regions 33 of the forefoot portion 26 that extend from the distal region 46 of the forefoot portion 26. The entire continuous rim 56 of the boot 10 surrounding the anterior opening 36 may be formed with stitching and/or a low-friction fabric trim that is sewn onto the shell 12 so as to form the rim 56 by compressing and narrowing the foam material along the entire perimeter of the anterior opening 36, as represented in FIGS. 2, 3, 5, and 6.

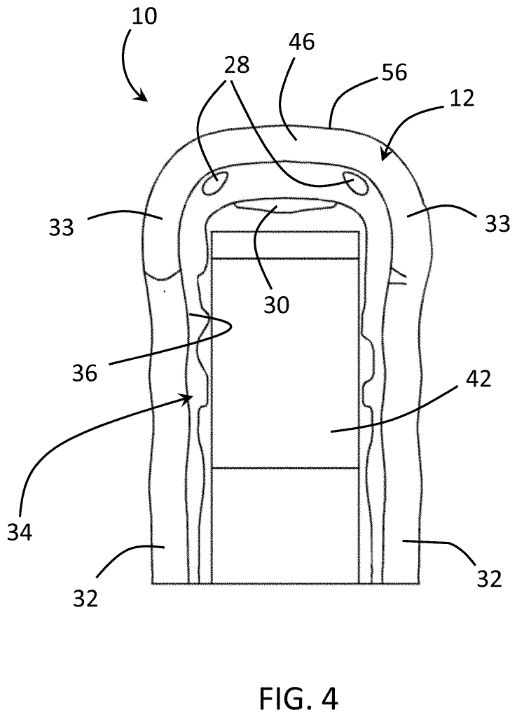

As most readily apparent in FIG. 6, the leg portion 24 of the shell 12 is adapted to support the lower leg 16 and elevate the foot 14 of an individual, and the forefoot portion 26 supports the foot 14 and, through the distal region 46 of the forefoot portion 26, applies supportive pressure to the foot sole for the purpose of preventing foot-drop (and equinus contractures). As previously noted, the leg portion 24 has left and right lateral regions 32, the forefoot portion 26 has left and right lateral regions 33, and together the leg and forefoot portions 24 and 26 define the continuous cavity 34 within the boot 10. The anterior opening 36 to the cavity 34 is defined by and between the lateral regions 32 and 33, and is sized to permit an individual's foot 14 and lower leg 16 to be lowered therethrough into the cavity 34. By comparing FIGS. 2 and 3, it can be seen that the right lateral region 32 of the leg portion 24 is larger in size than the opposing left lateral region 32, though it is also within the scope of the invention that the left lateral region could be larger than the right lateral region 32. In the embodiment shown, the right lateral region 32 is of sufficient size to nearly or entirely close the portion of the anterior opening 36 within the leg portion 24 of the shell 12, and in so doing is folded onto and covers at least part and more preferably the entire anterior portion of an individual's lower leg 16 received within the cavity 34. However, a comparison of FIGS. 2 and 3 also shows that the left and right lateral regions 33 of the forefoot portion 26 are equal in size (or at least nearly so), and neither lateral region 33 is of sufficient size to be able to close the portion of the anterior opening 36 within the forefoot portion 26 or be folded onto and completely cover the entire dorsum of an individual's foot 14 received within the cavity 34. The closure system of the boot 10 is adapted to sufficiently close the anterior opening 36 with the lateral regions 32 and 33 to facilitate tightening and adjustment of the boot 10 on an individual's lower leg 16.

The leg and forefoot portions 24 and 26 of the shell 12 are represented in the drawings as optionally comprising ventilation holes 28 to promote the comfort of the individual when the boot 10 is worn for long periods, though these holes 28 could be eliminated for some applications. The boot 10 is also represented as having a heel through-hole 30 at the intersection of the leg and forefoot portions 24 and 26 of the shell 12 in order to reduce the likelihood that any portion of the boot 10 will contact the individual's heel 18. The heel through-hole 30 is preferably sufficiently small to prevent the heel 18 from passing therethrough. To maintain proper ventilation of the cavity 34, the ventilation holes 28 and heel through-hole 30 should remain uncovered by the cover materials 18 on the exterior surface of the shell 12, as well as any inner lining material at the interior surface of the boot 10.

In the nonlimiting embodiment shown, the closure system comprises a pair of leg straps 38 adapted to traverse the portion of the anterior opening 36 within the leg portion 24 of the shell 12. Each leg strap 38 works in cooperation with a buckle 50 attached to one of the lateral regions 32 of the leg portion 24. Each strap 38 may be attached to the lateral region 32 opposite its buckle 50 (e.g., the right lateral region 32 in the drawings), or optionally attached on the same lateral region 32 near the attachment point of its corresponding buckle 50 (e.g., the left lateral region 32 in the drawings). In use, the straps 38 are passed over the anterior opening 36 of the shell 12 toward their respective buckles 50, which provide generally D-shaped rings or slots through which the straps 38 can be inserted and then drawn back on themselves, allowing each strap 38 to be secured to itself with a suitable fastener, such as a complementary hook and loop closure material. As depicted in the drawings, the buckles 50 may be attached to the smaller lateral region 32 located on the left side of the boot 10, so that the larger lateral region 32 on the right side of the boot 10 is drawn down over the leg 16 as the straps 38 are passed over the anterior opening 36 toward the buckles 50. By inserting the straps 38 in the buckles 50 and then drawing the straps 38 back onto themselves, the larger lateral region 32 is drawn snug over the individual's lower leg 16, as can be appreciated from FIGS. 1 and 6.

The closure system further includes a foot strap 40 and corresponding buckle 52 attached to the exterior surface of the forefoot portion 26. The foot strap 40 spans the portion of the anterior opening 36 within the forefoot portion 26 and is adapted to adjustably narrow the anterior opening 36 within the forefoot portion 26, enabling the forefoot portion 26 and its lateral regions 33 to be brought into contact with and secured about the foot 14. Because of their equal size, the lateral regions 33 of the forefoot portion 26 apply approximately equal support and pressure to the lateral sides of the foot 14. As with the buckles 50 of the straps 38, the buckle 52 associated with the foot strap 40 may be a generally D-shaped ring or slot through which the strap 40 can be inserted and then drawn back on itself, allowing the strap 40 to be secured to itself with a suitable fastener, such as a complementary hook and loop closure material. As portrayed in FIG. 1, the forefoot portion 26, its lateral regions 33, and its strap 40 are preferably configured so that the strap 40 does not cause the lateral regions 33 to completely close the anterior opening 36 within the forefoot portion 26, but instead draws the lateral regions 33 inward toward each other and into contact with the lateral sides of the foot 14. Because of the equal size the lateral regions 33, the strap 40 can be used to cause the lateral regions 33 to apply approximately equal pressure and support to the left and right lateral sides of the foot 14. In so doing, the lateral regions 33 of the forefoot portion 26 are able to sufficiently support the left and right lateral sides of the foot 14 to provide resistance to movement of the foot 14 in the pronation and supination directions, while providing only minimal if any support to the dorsum of the foot 14. Instead, the forefoot portion 26 and its the lateral regions 33 are sized and configured so that the anterior opening 36 remains partially open after the strap 40 has drawn the lateral regions 33 inward toward each other and into contact with the lateral sides of the foot 14, and the strap 40 continues to span the incompletely closed anterior opening 36.

The closure system also includes a forefoot flap 44 that spans the incompletely closed anterior opening 36 within the forefoot portion 26 as well as the dorsum of a foot 14 within the forefoot portion 26. In the nonlimiting embodiment shown, the forefoot flap 44 extends from one of the lateral regions 33 of the forefoot portion 26, spans the incompletely closed anterior opening 36 within the forefoot portion 26, and extends to (and optionally overlaps) the other lateral region 33 of the forefoot portion 26. As seen in FIGS. 1 and 6, the foot strap 40 traverses the exterior surface of the forefoot flap 44, and as depicted in FIG. 6 the flap 44 also preferably contacts the dorsum of the foot 14 to apply limited pressure to the dorsum of the foot. As such, the foot strap 44 is between the strap 40 and foot 14 as the strap 40 spans the anterior opening 36 so that the strap 40 does not contact the foot 14. The inclusion of the forefoot flap 44 protects the dorsum of the foot 14 from the strap 40, but also enables the strap 40 and the forefoot portion 26 as a whole to apply pressure to the dorsum and, in combination with the pressure applied to the sole of the foot 14 with the foot strap 40 via the distal region 46 of the forefoot portion 26, substantially increase resistance to movement of the foot 14 in the dorsiflexion and plantarflexion directions. Importantly, the forefoot flap 44 achieves this benefit while also distributing the pressure applied by the strap 40 over the skin region of the foot 14 contacted by the flap 44 to ensure that the pressure to which the dorsum of the foot 14 is subjected does not exceed 32 mm Hg, which would compromise the capillary circulation and pcO₂ (partial pressure of oxygen) in the tissue of the dorsum and possibly the foot 14 as a whole. For this purpose, the forefoot flap 44 must have a greater surface area within the anterior opening 36 than does the strap 40. In practice, a suitable width for the flap 44 is on the order of about 10 cm or more and a suitable size (surface area) for the flap 44 is on the order of about 100 cm² or more, though smaller and larger flaps 44 are foreseeable. In order to distribute pressure and protect the dorsum from the strap 40, the forefoot flap 44 should also exhibit sufficient rigidity. For this purpose, a discrete stiffener member may be incorporated into the flap 44, though in practice sufficient rigidity is achieved by forming the flap 44 from the same open-cell foam material as used to make the shell 12. The inclusion of the forefoot flap 44 may also further enable the lateral regions 33 of the forefoot portion 26 to resist movement of the foot 14 in the pronation and supination directions. In any case, the forefoot flap 44 helps to protect the wearer from shear and friction forces that might damage the skin, and is effective whether the wearer is in the supine position (reclining), the prone position (face down), transitioning between these two positions, reclining on a bed or wheelchair, etc.

The boot 10 further comprises a cushion 42 (FIGS. 4 and 6) within the shell cavity 34 for further elevating the lower leg 16 and supporting the foot 14 and lower leg 16 without applying any support pressure at the heel 18 of an individual's foot 14, thereby avoiding pressure sores at the heel 18. As depicted in FIG. 6, the cushion 42 may be a separable and adjustable cushion 42, though a cushion 42 that is integral with the shell 12 is also within the scope of the invention. The combination of the cushion 42 and a firmer forefoot portion 26 (due to the forefoot flap 44) is believed to improve the ability of the boot 10 to resist movement of the foot 14 in the dorsiflexion and plantarflexion directions, as well as the pronation and supination directions, and to protect the forefoot from pressure injuries.

From the foregoing, it should be appreciated that the boot 10 provides several advantageous structural adaptations. The lateral regions 33 of the forefoot portion 26 are sized and configured to contact and provide approximately equal support to the left and right lateral sides of the foot 14, whereas the forefoot flap 44 of the forefoot portion 26 serves to contact and provide support to the dorsum of the foot 14, which in combination provides greater support to the foot 14 that is capable of resisting movement of the foot 14 in the dorsiflexion and plantarflexion directions, as well as the pronation and supination directions.

While the invention has been described in terms of a particular embodiment, it should be apparent that alternatives could be adopted by one skilled in the art. For example, the boot 10 and its components could differ in appearance and construction from the embodiment described herein and shown in the drawings, functions of certain components of the boot 10 could be performed by components of different construction but capable of a similar (though not necessarily equivalent) function, and appropriate materials could be substituted for those noted. As such, it should be understood that the intent of the above detailed description is to describe the particular embodiment represented in the drawings and certain but not necessarily all features and aspects thereof, and to identify certain but not necessarily all alternatives to the particular embodiment represented in the drawings. As a nonlimiting example, the invention encompasses additional or alternative embodiments in which one or more features or aspects of the described embodiment could be eliminated. Accordingly, it should be understood that the invention is not necessarily limited to any particular embodiment represented in the drawings or described herein, and that the purpose of the above detailed description and the phraseology and terminology employed therein is to describe the particular embodiment represented in the drawings, and not necessarily to serve as limitations to the scope of the invention. Therefore, the scope of the invention is to be limited only by the following claims.

1. A protective boot configured to be worn on a human foot and lower leg of an individual while reclining to support a lower leg, heel, and foot of the individual while reclining to reduce a risk of foot drop and heel pressure ulcers, the boot comprising:
a leg portion and a forefoot portion contiguous with and projecting from the leg portion, the forefoot portion having a distal region and oppositely-disposed first and second forefoot lateral regions that extend from the distal region of the forefoot portion;
a continuous cavity within the leg and forefoot portions of the boot, the cavity being complementary in size and shape to receive and support the lower leg and the foot of the individual while supporting the foot of the individual in an upright position;
a rim that borders an anterior opening to the cavity including the first and second forefoot lateral regions of the forefoot portion, a portion of the anterior opening within the forefoot portion being between the first and second forefoot lateral regions, the first and second forefoot lateral regions of the forefoot portion being sized so that neither of the first and second forefoot lateral regions is of sufficient size to be able to entirely close the portion of the anterior opening within the forefoot portion or to be folded onto each other to entirely close the portion of the anterior opening within the forefoot portion or completely cover an entirety of a dorsum of the foot of the individual within the pcavity;
an individual forefoot flap extending from the first forefoot lateral region and extending to the second forefoot lateral region, spanning the portion of the anterior opening in the forefoot portion without overlapping the second forefoot lateral region; and
a strap connecting the first and second forefoot lateral regions of the forefoot portion, entirely spanning the portion of the anterior opening in the forefoot portion spanned by the forefoot flap, overlapping the forefoot flap, and traversing an exterior surface of the forefoot flap so that the forefoot flap contacts the dorsum of the foot, separates and protects the dorsum from the strap, distributes pressure applied by the strap to the dorsum and, in combination with a distal region of the forefoot portion, resists movement of the foot in the dorsiflexion and plantarflexion directions.

2. The protective boot of claim 1, wherein the first and second forefoot lateral regions are equal in size.

3. A protective boot configured to be worn on a human foot and lower leg of an individual while reclining to support a lower leg, heel, and foot of the individual while reclining to reduce a risk of foot drop and heel pressure ulcers, the boot comprising:
a shell formed of a flexible and compressible material, the shell having a proximal leg portion, a distal forefoot portion contiguous with and projecting from the leg portion in a transverse direction thereto, a continuous cavity defined by and within the leg and forefoot portions and complementary in size and shape to receive and support the lower leg and the foot of the individual while supporting the foot of the individual in an upright position, a rim that borders an anterior opening defined in the leg and forefoot portions to permit the foot and lower leg of the individual to pass therethrough into the cavity within the leg and forefoot portions, left and right leg lateral regions defined by the leg portion and separated by a first portion of the anterior opening, and oppositely-disposed first and second forefoot lateral regions defined by the forefoot portion and separated by a second portion of the anterior opening, the first and second forefoot lateral regions of the forefoot portion being sized so that neither of the first and second forefoot lateral regions is of sufficient size to be able to entirely close the portion of the anterior opening within the forefoot portion or to be folded onto each other to entirely close the portion of the anterior opening within the forefoot portion or completely cover an entirety of a dorsum of the foot of the individual within the cavity;
a closure system comprising at least a first strap that adjustably closes the first portion of the anterior opening with the left and right leg lateral regions of the leg portion and at least a second strap that adjustably draws the first and second forefoot lateral regions of the forefoot portion toward each other across the second portion of the anterior opening so as to enable the first and second forefoot lateral regions of the forefoot portion to contact and support lateral sides of the foot of the individual when present within the cavity and resist movement of the foot in the pronation and supination directions thereof but provide only minimal if any support to the dorsum of the foot; and
an individual forefoot flap that extends from the first forefoot lateral region and extends to the second forefoot lateral region without overlapping the second forefoot lateral region, spans the second portion of the anterior opening within the forefoot and overlaps the dorsum of the foot of the individual wearing the boot, the second strap of the closure system connecting the first and second forefoot lateral regions of the forefoot portion, entirely spanning the second portion of the anterior opening in the forefoot portion spanned by the forefoot flap, overlapping the forefoot flap, and traversing an exterior surface of the forefoot flap so that the forefoot flap is enabled to contact the dorsum of the foot when present within the cavity, separate and protect the dorsum of the foot from the second strap, distribute pressure applied by the second strap to the dorsum and, in combination with pressure applied to a sole of the foot by a distal region of the forefoot portion, resist movement of the foot in the dorsiflexion and plantarflexion directions.

4. The protective boot of claim 3, wherein one of the left and right leg lateral regions is larger in size than the other.

5. The protective boot of claim 4, wherein the one of the left and right leg lateral regions is of sufficient size to close the first portion of the anterior opening within the leg portion of the shell.

6. The protective boot of claim 3, wherein the first and second forefoot lateral regions are equal in size.

7. The protective boot of claim 6, wherein neither of the first and second forefoot lateral regions closes any part of the second portion of the anterior opening within the forefoot portion.

8. The protective boot of claim 3, wherein the forefoot portion and the first and second forefoot lateral regions are sized and configured so that the second portion of the anterior opening remains partially open after the second strap of the closure system has drawn the first and second forefoot lateral regions toward each other and the second strap spans the second portion of the anterior opening.

9. The protective boot of claim 3, wherein the forefoot flap applies a pressure not exceeding 32 mm Hg to the dorsum of the foot of the individual.

10. The protective boot of claim 3, wherein the forefoot flap has a greater surface area within the second portion of the anterior opening than does the second strap.

11. The protective boot of claim 3, wherein the forefoot flap has a width of 10 centimeters or more.

12. The protective boot of claim 11, wherein the forefoot flap has a surface area of 100 cm2 or more.

13. The protective boot of claim 3, wherein the forefoot flap is formed of the flexible and compressible material of the shell.

14. A method of using the protective boot of claim 3, the method comprising:
receiving and supporting the lower leg and the foot of the individual within the continuous cavity within the leg and forefoot portions so as to support the foot of the individual in an upright position within the forefoot portion;
spanning the second portion of the anterior opening and the dorsum of the foot with the forefoot flap; and
traversing the exterior surface of the forefoot flap with the second strap of the closure system and drawing the first and second forefoot lateral regions toward each other with the second strap so that the second strap applies pressure to the forefoot flap and the forefoot flap contacts the dorsum of the foot, separates and protects the dorsum of the foot from the second strap, distributes the pressure applied by the second strap to the dorsum and, in combination with the pressure applied to the sole of the foot by the distal region of the forefoot portion, resists movement of the foot in the dorsiflexion and plantarflexion directions.

15. The method of claim 14, further comprising traversing the first portion of the anterior opening with the first strap of the closure system and closing the first portion of the anterior opening with at least one of the left and right leg lateral regions of the leg portion.

16. The method of claim 15, wherein only one of the left and right leg lateral regions closes the first portion of the anterior opening within the leg portion.

17. The method of claim 14, wherein neither of the first and second forefoot lateral regions closes the second portion of the anterior opening within the forefoot portion after the second strap of the closure system has drawn the first and second forefoot lateral regions toward each other.

18. The method of claim 14, wherein the second portion of the anterior opening remains partially open after the second strap of the closure system has drawn the first and second forefoot lateral regions toward each other.

19. The method of claim 14, wherein the pressure applied by the forefoot flap to the dorsum of the foot does not exceed 32 mm Hg.

20. The method of claim 14, wherein the forefoot flap has a greater surface area within the second portion of the anterior opening than does the second strap.
